(19) United States
(12) Patent Application Publication
(10) Pub. No.: US 2005/0250957 A1
(43) Pub. Date: Nov. 10, 2005

(54) Compounds containing thiosulfate moieties

(76) Inventors: Richard Haugland, Eugene, OR (US); Hee Chol Kang, Eugene, OR (US)

Correspondence Address: KOREN ANDERSON;MOLECULAR PROBES, INC., 29851 WILLOW CREEK ROAD, EUGENE, OR, 97402-9132, US

(21) Appl. No.: 10/982,301

(22) Filed: Nov. 5, 2004

Related U.S. Application Data
(60) Provisional application No. 60/518,151, filed on Nov. 7, 2003.

(51) Int. Cl. C07C 305/00

(52) U.S. Cl. 558/31

(57) ABSTRACT

The present invention provides chemical compounds containing a thiosulfate group, and methods for their preparation and use. The thiosulfate group reacts with a thiol group to form a disulfide bond.

### CROSS REFERENCE TO RELATED APPLICATIONS

The present application claims priority to U.S. Provisional Patent Application Ser. No. 60/518,151 filed Nov. 7, 2003, the contents of which are incorporated herein by reference.

### FIELD OF THE INVENTION

The present invention relates to thiosulfate containing compounds, and methods for their preparation and use. The compounds have applications in a variety of fields such as immunology, diagnostics, molecular biology and fluorescence based assays.

### DESCRIPTION OF RELATED ART

Chemical compounds that function as stains or dyes have been used for many years. Typically, the compounds form an irreversible covalent bond with a target molecule, and confer a detectable property onto the labeled target. The detection can be performed using one of various visualization techniques.

A variety of visualization techniques are known in the art. Optical methods of detection, such as fluorescence emission, UV absorption, and colorimetry are convenient and highly effective for detecting, monitoring, and measuring the presence of a labeled target. Detection methods such as these can generate either qualitative or quantitative information and detection can be achieved either by direct visual observation or by instrumentation. Optically detectable reporters, i.e., synthetic or substitute substrates that are added to a sample and that display a measurable increase or other difference in optical detectability upon action of the enzyme, are therefore particularly useful. Examples of optical reporters that are currently known are 4-nitrophenol, α-naphthol, β-naphthol, resorufin and substituted resorufins, nitranilide, 5-bromo-4-chloro-3-indole, and umbelliferone derivatives. The degree of change and hence the effectiveness of optical detection reporters depend on any of several factors, depending on the detection method for which they are used. Some of these factors are, a high extinction coefficient for reporters that are detectable by light absorptivity (particularly a large increase from substrate to product), a large change in the wavelength at which maximum absorptivity occurs (particularly a large substrate-to-product red shift), a substrate-to-product increase in the Stokes' shift for fluorescent reporters, and the chemical stability of the reporter.

With the advent of nanotechnology, there is an increased ability to perform numerous chemical and physical operations with very small volumes. This opportunity comes with the requirement that determinations have enhanced sensitivity to detect the fewer molecules that are present to provide the detectable signal. Part of the increased sensitivity may come from more sensitive detectors, but these are usually more expensive and are not readily available in most laboratories. The other opportunity is to provide assays that are more efficient in providing for detectable products, and provide products with a strong signal (e.g., a high emission efficiency for fluorescent compounds).

Thus, there is a need in the art for superior reagents capable of covalently binding to targets. It would be additionally attractive to develop reagents that can reversibly covalently bond to targets. The present invention provides compounds and methods that fill these and other needs.

### SUMMARY OF THE INVENTION

The present invention provides a novel class of compounds for use in a wide variety of biological, chemical, and biochemical methods such as target labelling, target analyte detection, crosslinking reactions, and purification techniques. The novel compounds contain a thiosulfate moiety that is capable of reacting with a thiol moiety to form a disulfide bond. Methods for the preparation of compounds containing the thiosulfate moiety are also disclosed.

Embodiments of the invention generically include compounds of the structure R—S—SO₃⁻ (R—S₂O₃⁻). The remainder of the compound (R) is covalently bonded to the thiosulfate group through the central sulfur atom. The R group can generally be any type of compound. Presently preferred embodiments include the R group being a carrier molecule, a reporter group, or a solid support. The compounds can also include a linker group L, positioned between R and the thiosulfate group, covalently attached to each.

The thiosulfate moiety reacts with a thiol group to form a disulfide bond. The thiol group can be present on a different molecule, leading to an intermolecular reaction, or can be present on the same molecule, leading to an intramolecular reaction. The formation of the disulfide bond is reversible, that is, the disulfide bond can react with an agent to cleave the disulfide bond. This feature is quite distinct from other chemistries commonly used in the art.

The compounds can be used in a variety of methods such as labeling a target, purifying thiol-containing materials from a mixture, crosslinking a material, immobilizing a thiol-containing material on a solid support.

Alternative embodiments include kits containing a thiosulfate compounds. The kits can also include instructions for performing one or more methods using the included compounds.

### DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1A-B show synthetic methods for preparing thiosulfate compounds.

FIGS. 2A-C show synthetic methods for preparing thiosulfate compounds.

FIG. 3 shows the reaction of a boradiazaindacene thiosulfate with a thiol.

FIG. 4 shows the reaction of a boradiazaindacene thiosulfate with a protein.

### DEFINITIONS

The following definitions are provided in order to aid those skilled in the art in understanding the detailed description of the present invention.

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a target analyte” includes a plurality of proteins and reference to “a thiosulfate compound” includes a plurality of thiosulfate compounds and the like.

Unless defined otherwise, all technical and scientific terms used herein have the same meaning as commonly understood by one of ordinary skill in the art to which this invention is related. The following terms are defined for purposes of the invention as described herein.

The “squiggly line” symbol [symbol] , whether used as a bond or displayed perpendicular to a bond indicates the point at which the displayed moiety is attached to the remainder of the molecule, solid support, etc.

Certain compounds of the present invention can exist in unsolvated forms or salts as well as solvated forms, including hydrated forms. In general, the solvated forms and salts are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention. Salts can generally include any appropriate counterion. For example, a negatively charged thiosulfate compound could have a group I (alkali) counterion, a group II (alkaline earth) counterion, ammonium counterion, polyalkylammonium counterion, or other metal or organic cations. Alkali counterions can include sodium, potassium, or others. Alkaline earth counterions can include calcium, magnesium, or others. Transition group metal counterions can include iron, copper, nickel, silver, or others.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5TH ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., CH₂O is intended to also recite —OCH₂.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid lacking the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C1-C10 means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Similarly, the term “alkylene” by itself or as part of another substituent means a divalent radical derived from alkyl. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P and S, and wherein the nitrogen, phosphorous and sulfur atoms are optionally oxidized, and the nitrogen heteroatom is optionally be quaternized. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linkers, no orientation of the linker is implied by the direction in which the formula of the linker is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5′,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. Similarly, the terms “cycloalkylene” or “heterocycloalkylene” by itself or as part of another substituent means a divalent radical derived from cycloalkyl or heterocycloalkyl, respectively.

The term “aryl” means, unless otherwise stated, a polyunsaturated, aromatic moiety that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems are selected from the group of acceptable substituents described below. Similarly, the terms “arylene” or “heteroarylene” by itself or as part of another substituent means a divalent radical derived from aryl or heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) is meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, OC(O)R′, —C(O)R′, —CO2R′, —CONR′R″, —OC(O)NR′R″, —NR″C(O)R′, NR′C(O)NR″R′″, —NR″C(O)2R′, —NR—C(NR′R″R′″)═NR″″, NRC(NR′R″)═NR′″, —S(O)R′, —S(O)2R′, —S(O)2NR′R″, NRSO2R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, OR′, ═O, ═NR′, ═N—OR′, —NR′R″, —SR′, -halogen, —SiR′R″R′″, OC(O)R′, —C(O)R′, CO2R′, —CONR′R″, —OC(O)NR′R″, —NR″C(O)R′, NR′ C(O)NR″R′″, —NR″C(O)2R′, NR—C(NR′R″R′″)═NR″″, NRC(NR′R″)═NR′″, —S(O)R′, —S(O)₂R′, —S(O)₂NR′R″, NRSO₂R′, —CN and —NO₂, —R′, —N3, —CH(Ph)2, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)q-U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A(CH2)rB—, wherein A and B are independently —CRR′—, —O—, —NR—, —S—, —S(O)—, S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “heteroatom” includes oxygen (O), nitrogen (N), sulfur (S), phosphorus (P), and silicon (Si).

The term “amino” or “amine group” refers to the group —NR′R″ (or N⁺RR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine is an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N⁺RR′R″ and its biologically compatible anionic counterions.

The term “aqueous solution,” as used herein, refers to a solution that is predominantly water and retains the solution characteristics of water. Where the aqueous solution contains solvents in addition to water, water is typically the predominant solvent.

The term “attachment site” as used herein refers to a site on a moiety or a molecule to which it is covalently attached, or capable of being covalently attached, to a linker or another moiety. For example, a first molecule can have a primary amine group capable of being covalently attached to a carboxylic acid group on a second molecule.

The term “carrier molecule” as used herein refers to a fluorogenic, fluorescent or colorimetric compound of the present invention that is covalently bonded to a biological or a non-biological component. Such components include, but are not limited to, an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof.

The term “detectable response,” as used herein, refers to a change in or an occurrence of, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters. Compounds can impart detectable responses on their own (e.g., a dye), or can impart detectable responses upon interaction with another compound or upon encountering a particular environment (e.g., a stain).

The term “dye” as used herein refers to a compound that absorbs light beyond about 300 nm. “Dye” includes fluorescent and nonfluorescent compounds that include without limitations pigments, fluorophores, chemiluminescent compounds, luminescent compounds and chromophores. The term “fluorophore” as used herein refers to a composition that is inherently fluorescent or demonstrates a change in fluorescence upon binding to a biological compound or metal ion, or metabolism by an enzyme, i.e., is fluorogenic. Fluorophores may be substituted with one or more groups to alter the solubility, spectral properties or physical properties of the fluorophore. Numerous fluorophores are known to those skilled in the art and include, but are not limited to benzofurans, quinolines, quinazolinones, indoles, benzazoles, borapolyazaindacenes and xanthenes, with the latter including fluoresceins, rhodamines and rhodols as well as other fluorophores described in RICHARD P. HAUGLAND, MOLECULAR PROBES HANDBOOK OF FLUORESCENT PROBES AND RESEARCH CHEMICALS (9th edition, including the CD-ROM, September 2002).

The term “enzyme” as used herein refers to a protein molecule produced by living organisms, or through chemical modification of a natural protein molecule, that catalyzes a chemical reaction of other substances without itself being destroyed or altered upon completion of the reactions. Examples of other substances, include, but are not limited to chemiluminescent, chromogenic and fluorogenic substances or protein-based substrates.

The term “fluorescent or fluorogenic labeled component,” as used herein, refers to a fluorescent or fluorogenic compound that is covalently bonded to a biological or a non-biological component. Such components include, but are not limited to, an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “linker” or “L” as used herein refers to a single covalent bond or a series of stable covalent bonds incorporating 1-20 nonhydrogen atoms selected from the group consisting of C, N, O, S and P that covalently attach the present quenching compounds to another moiety such as a chemically reactive group or a conjugated substance including biological and non-biological substances. Exemplary linking members include —C(O)NH, C(O)O, NH, S, O, and the like. A “cleavable linker” is a linker that has one or more cleavable groups that may be broken by the result of a reaction or condition. The term “cleavable group” refers to a moiety that allows for release of a portion, e.g., a fluorogenic or fluorescent moiety, of a conjugate from the remainder of the conjugate by cleaving a bond linking the released moiety to the remainder of the conjugate. Such cleavage is either chemical in nature, or enzymatically mediated. Exemplary enzymatically cleavable groups include natural amino acids or peptide sequences that end with a natural amino acid. An example of a chemically cleavable group (or linker) is a disulfide group (—S—S—). Disulfide groups are distinctive in that they are covalent linkers, yet are readily cleaved by using free thiols, phosphines, or other agents. Disulfides, once cleaved, release a free thiol group from which it was formed. Many other linkers leave a small portion covalently attached after leavage. Additionally, most linkers are relatively or completely irreversible once formed.

In addition to enzymatically cleavable groups, it is within the scope of the present invention to include one or more sites that are cleaved by the action of an agent other than an enzyme. Exemplary non-enzymatic cleavage agents include, but are not limited to, acids, bases, light (e.g., nitrobenzyl derivatives, phenacyl groups, benzoin esters), and heat. Many cleaveable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover a broad range of cleavable, bifunctional (both homo- and hetero-bifunctional) spacer arms are commercially available.

An exemplary cleavable group, an ester, is cleavable group that may be cleaved by a reagent, e.g. sodium hydroxide or hydroxylamine, resulting in a carboxylate-containing fragment and a hydroxyl-containing product.

“Non-covalent protein binding groups” are moieties that interact with an intact or denatured polypeptide in an associative manner. The interaction may be either reversible or irreversible in a biological milieu. The incorporation of a “non-covalent protein binding group” into a fluorogenic compound provides the compound with the ability to interact with a polypeptide in a non-covalent manner. Exemplary non-covalent interactions include hydrophobic-hydrophobic and electrostatic interactions. Exemplary “non-covalent protein binding groups” include anionic groups, e.g., phosphate, thiophosphate, phosphonate, carboxylate, boronate, sulfate, sulfone, thiosulfate, and thiosulfonate.

The term “protein tag binder” is a non-covalent protein binding group that binds to a protein tag. Protein tag binders preferably bind their protein tag binding partners in a substantially specific manner. Antigens may also serve as protein tag binders as they are capable of binding antibodies. A receptor that binds a protein ligand is another example of a possible protein tag binder. Protein tag binders as used herein are understood to be limited to agents, that only interact with their binding partners through noncovalent interactions.

The term “protein tag” means that portion of a protein that is bound by a particular protein tag binder, preferably in a substantially specific manner. In some cases, the binding partner or tag may be the protein normally bound in vivo by a protein, which is a protein tag binder (e.g., antibody-antigen binding pairs). Additionally, the protein tag or binding partner may be the protein or peptide on which the protein tag binder was selected (through in vitro or in vivo selection) or raised (as in the case of antibodies). A binding partner may be shared by more than one protein tag binder. For instance, a binding partner that is bound by a variety of polyclonal antibodies may bear a number of different epitopes. One protein tag binder may also bind to a multitude of binding partners (for instance, if the binding partners share the same epitope). In view of the above, the terms “protein tag” and “protein tag binder” is meant to include, but not be limited to, those pairs such as fusion tags/tag binders, protein/ligand, enzyme/substrate, and antibody/antigen.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a quencher, a fluorophore or another moiety.

“Peptide” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

“Protecting group,” as used herein refers to a portion of a substrate that is substantially stable under a particular reaction condition, but which is cleaved from the substrate under a different reaction condition. For examples of useful protecting groups, see, for example, Greene et al., PROTECTIVE GROUPS IN ORGANIC SYNTHESIS, John Wiley & Sons, New York, 1991.

The term “reactive group” as used herein refers to a group that is capable of reacting with another chemical group to form a covalent bond, i.e. is covalently reactive under suitable reaction conditions, and generally represents a point of attachment for another substance. The reactive group is a moiety, such as carboxylic acid or succinimidyl ester, that is capable of chemically reacting with a functional group on a different compound to form a covalent linkage. Reactive groups generally include nucleophiles, electrophiles and photoactivatable groups.

Exemplary reactive groups include, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “reporter group” as used herein refers to a chemical moiety or protein that has attractive properties (e.g., spectral properties, conformation and activity) when attached to a thiosulfate group and used in the present methods. The reporter group can be directly detectable (dye) or indirectly detectable (hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent labels (fluorophores). The reporter group can be a chemiluminescent substance, where the output signal is generated by chemical modification of the signaling compound; a metal-containing substance; or an enzyme, where there occurs an enzyme-dependent secondary generation of signal, such as the formation of a colored product from a colorless substrate. The term reporter molecule can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidase (HRP) to bind to the tag, and then use a colorimetric substrate (e.g., tetramethylbenzidine (TMB)) or a fluorogenic substrate such as Amplex Red reagent (Molecular Probes, Inc.) to detect the presence of HRP. Numerous reporter group are known by those of skill in the art and include, but are not limited to, particles, dyes, haptens, enzymes and their colorimetric, fluorogenic and chemiluminescent substrates and other reporter groups that are described in RICHARD P. HAUGLAND, MOLECULAR PROBES HANDBOOK OF FLUORESCENT PROBES AND RESEARCH PRODUCTS (9th edition, CD-ROM, September 2002), supra.

The term “sample” as used herein refers to any material that may contain a target analyte. The sample may also include diluents, buffers, detergents, and contaminating species, debris and the like that are found mixed with the target. Illustrative examples include urine, sera, blood plasma, total blood, saliva, tear fluid, cerebrospinal fluid, secretory fluids from nipples and the like. Also included are solid, gel or sol substances such as mucus, body tissues, cells and the like suspended or dissolved in liquid materials such as buffers, extractants, solvents and the like. The sample can be a live cell, a biological fluid that comprises endogenous host cell proteins, nucleic acid polymers, nucleotides, oligonucleotides, peptides and buffer solutions. The sample may be in an aqueous solution, a viable cell culture or immobilized on a solid or semi solid surface such as a polyacrylamide gel, membrane blot or on a microarray.

“Solid support,” as used herein, refers to a material that is substantially insoluble in a selected solvent system, or that can be readily separated (e.g., by precipitation) from a selected solvent system in which it is soluble. Solid supports useful in practicing the present invention can include groups that are activated or capable of activation to allow selected species to be bound to the solid support. Solid supports may be present in a variety of forms, including a chip, wafer or well, paper, glass, or plastic onto which an individual, or more than one compound, of the invention is bound. The solid support can have a variety of shapes such as flat (e.g., a plate or chip) or round (e.g., a spherical bead).

A “target analyte,” as used herein, means any microorganism, compound or molecule of interest for which detection is desired. A target analyte can be, for example, a protein, peptide, carbohydrate, liposome, fatty acid, phospholipid, triacyl glycerol, polysaccharide, glycoprotein, hormone, receptor, antigen, antibody, pathogenic microorganism or virus, substrate, pharmaceutical, metabolite, narcotic, poison, transition state analog, cofactor, inhibitor, dye, nutrient, growth factor, antibodies, enzymes, and derivatives and combinations thereof, without limitation. The term can refer to a single component or a plurality of components in a given sample.

The term “targeting group” refers to a moiety that is: (1) able to actively direct the entity to which it is attached (e.g., a fluorogenic moiety) to a target region, e.g., a cell; or (2) is preferentially passively absorbed by or entrained within a target region. The targeting group can be a small molecule, which is intended to include both non-peptides and peptides. The targeting group can also be a macromolecule, which includes, but is not limited to, saccharides, lectins, receptors, ligands for receptors, proteins such as BSA, antibodies, poly(ethers), dendrimers, poly(amino acids) and so forth.

### DETAILED DESCRIPTION OF THE INVENTION

While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

Compounds

Embodiments of the present invention provide thiosulfate compounds comprising at least one thiosulfate group (i.e., —S—SO₃H or —S—SO₃⁻). Excluded are naturally ocurring thiosulfate ions and salts (S₂O₃²⁻; such as ammonium thiosulfate, potassium thiosulfate, barium thiosulfate, and calcium thiosulfate). The thiosulfate group is covalently bonded through its thio function (—S—) to one of a variety of components (Z-). The component Z can generally be any type of molecule. Typically, the Z group is a carrier molecule, a reporter group, a solid support, a reactive group, or a combination thereof. The thiosulfate compounds can generally have the structure Z-S—SO₃H or its conjugate base Z-S—SO₃—. The structure can further be represented in shorthand by Z-S₂O₃H or its conjugate base Z-S₂O₃—. The Z group can further contain one or more additional reactive groups. The compounds can contain one thiosulfate group, or multiple thiosulfate groups. For example, the compounds can have 2, 3, 4, 5, 6, 7, 8, 9, 10, or more thiosulfate groups.

A thiosulfate group, as used herein, refers to the group —S—SO₃H, its conjugate base —S—SO₃⁻, and salts thereof. The thio function of the thiosulfate moiety refers to the thio portion (—S—), rather than the sulfate portion (—SO₃H) of the thiosulfate group. Thus, a thiosulfate group covalently bonded to something through its thio function refers to a thiosulfate group attached through the thio portion (—S—) of the thiosulfate group. Thiosulfate groups can react with a thiol group (—SH) to form a disulfide bond. This reaction can be intramolecular or intermolecular.

The Z group can comprise a carrier molecule. Exemplary carrier molecules include antigens, steroids, vitamins, drugs, haptens, metabolites, toxins, environmental pollutants, amino acids, peptides, proteins, nucleic acids, nucleic acid polymers, carbohydrates, lipids, and polymers. In another embodiment, the carrier molecule is an amino acid, peptide, protein, polysaccharide, nucleotide, nucleoside, oligonucleotide, nucleic acid, hapten, psoralen, drug, hormone, lipid, phospholipid, lipoprotein, lipopolysaccharide, liposome, lipophilic polymer, synthetic polymer, polymeric microparticle, biological cell, virus, and combinations thereof.

In an exemplary embodiment, the carrier molecule comprises an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof. In another exemplary embodiment, the carrier molecule is selected from a hapten, a nucleotide, an oligonucleotide, a nucleic acid polymer, a protein, a peptide or a polysaccharide.

In another exemplary embodiment, the carrier molecule is an amino acid (including those that are protected or are substituted by phosphates, carbohydrates, or C₁ to C₂₂ carboxylic acids), or a polymer of amino acids such as a peptide or protein. In a related embodiment, the carrier molecule contains at least five amino acids, more preferably 5 to 36 amino acids. Exemplary peptides include, but are not limited to, neuropeptides, cytokines, toxins, protease substrates, and protein kinase substrates. Other exemplary peptides may function as organelle localization peptides, that is, peptides that serve to target the conjugated compound for localization within a particular cellular substructure by cellular transport mechanisms. Preferred protein carrier molecules include enzymes, antibodies, lectins, glycoproteins, histones, albumins, lipoproteins, avidin, streptavidin, protein A, protein G, phycobiliproteins and other fluorescent proteins, hormones, toxins and growth factors. Typically, the protein carrier molecule is an antibody, an antibody fragment, avidin, streptavidin, a toxin, a lectin, or a growth factor. Exemplary haptens include biotin, digoxigenin and fluorophores.

In another exemplary embodiment, the carrier molecule comprises a nucleic acid base, nucleoside, nucleotide or a nucleic acid polymer, optionally containing an additional linker or spacer for attachment of a fluorophore or other ligand, such as an alkynyl linkage (U.S. Pat. No. 5,047,519), an aminoallyl linkage (U.S. Pat. No. 4,711,955) or other linkage. In another exemplary embodiment, the nucleotide carrier molecule is a nucleoside or a deoxynucleoside or a dideoxynucleoside.

Exemplary nucleic acid polymer carrier molecules are single- or multi-stranded, natural or synthetic DNA or RNA oligonucleotides, or DNA/RNA hybrids, or incorporating an unusual linker such as morpholine derivatized phosphates (AntiVirals, Inc., Corvallis Oreg.), or peptide nucleic acids such as N-(2-aminoethyl)glycine units, where the nucleic acid contains fewer than 50 nucleotides, more typically fewer than 25 nucleotides. The thiosulfate moieties are optionally attached either directly or through a linker to the carrier molecule via one or more purine or pyrimidine bases through an amide, ester, ether or thioether bond; or attached to the phosphate or carbohydrate by a bond that is an ester, thioester, amide, ether or thioether. Alternatively, the thiosulfate is attached by formation of a non-covalent association of the nucleic acid and a photoreactive linker, followed by illumination, resulting in a covalently bound thiosulfate moiety. Nucleotide carrier molecules of the invention can be incorporated by some DNA polymerases and can be used for in situ hybridization and nucleic acid sequencing (e.g., U.S. Pat. Nos. 5,332,666; 5,171,534; and 4,997,928; and PCT Publication WO 94/05688).

In another exemplary embodiment, the carrier molecule comprises a carbohydrate or polyol that is typically a polysaccharide, such as dextran, FICOLL (a registered trademark of Pharmacia Biotech, Uppsala, Sweden), heparin, glycogen, amylopectin, mannan, inulin, starch, agarose and cellulose, or is a polymer such as a poly(ethylene glycol). In a related embodiment, the polysaccharide carrier molecule includes dextran or FICOLL.

In another exemplary embodiment, the carrier molecule comprises a lipid (typically having about 6 to about 25 carbons), including glycolipids, phospholipids, and sphingolipids. Alternatively, the carrier molecule comprises a lipid vesicle, such as a liposome, or is a lipoprotein (see below). Some lipophilic substituents are useful for facilitating transport of the conjugated dye into cells or cellular organelles.

Alternatively, the carrier molecule is a cell, cellular systems, cellular fragment, or subcellular particles, including virus particles, bacterial particles, virus components, biological cells (such as animal cells, plant cells, bacteria, or yeast), or cellular components. Examples of cellular components that are useful as carrier molecules include lysosomes, endosomes, cytoplasm, nuclei, histones, mitochondria, Golgi apparatus, endoplasmic reticulum and vacuoles.

In another exemplary embodiment, the carrier molecule non-covalently associates with organic or inorganic materials. Exemplary embodiments of the carrier molecule that possess a lipophilic substituent can be used to target lipid assemblies such as biological membranes or liposomes by non-covalent incorporation of the dye compound within the membrane, e.g. for use as probes for membrane structure or for incorporation in liposomes, lipoproteins, films, plastics, lipophilic microspheres or similar materials.

The Z group can comprise a reporter group. The reporter group can generally be any reporter group that allows for the detection of the thiosulfate compound, either directly or indirectly. Examples of reporter groups include a fluorophore, a phosphorophore, a binding pair member, a peptide, a protein, a nucleic acid, an enzyme substrate, a radioisotope tag, a dye (including chromophores and fluorophores), a fluorescent protein, a phosphorescent dye, a tandem dye, a particle, a hapten, an enzyme, a spin label, a radioisotope, and combinations thereof.

A fluorophore is generally any chemical moiety that exhibits an absorption maximum beyond 280 nm, and has desirable spectral properties. Examples of fluorophores include a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine (including any corresponding compounds in U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; 6,664,047, and publications U.S. 2002-0064794 A1, U.S. 2002-0077487 A1, WO 02/26891, WO 97/40104, WO 99/51702, WO 01/21624; EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 5,451,343; 6,162,931; 6,130,101; 6,229,055; 6,339,392; 6,716,979), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980; and 5,830,912), a benzofuran (including an corresponding compounds disclosed in U.S. Pat. Nos. 4,603,209 and 4,849,362) and benzophenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

The fluorophore is optionally an ion-complexing moiety. While any chelator that binds an ion of interest and gives a change in its fluorescence properties is useful, preferred ion-complexing moieties are crown ethers, including diaryldiaza crown ethers, as described in U.S. Pat. No. 5,405,975; derivatives of 1,2-bis-(2-aminophenoxyethane)-N,N,N′,N′-tetraacetic acid (BAPTA), as described in U.S. Pat. No. 5,453,517; and U.S. Pat. No. 5,049,673; derivatives of 2-carboxymethoxy-aniline-N,N-diacetic acid (APTRA), as described by Ragu et al., Am. J. Physiol., 256: C540 (1989); and pyridyl-based and phenanthroline-based metal ion chelators, as described in U.S. Pat. No. 5,648,270. Fluorescent ion-complexing moieties possess utility as indicators for the presence of a desired metal ion. The ion-sensing molecules are optionally prepared in chemically reactive forms and further conjugated to polymers such as dextrans to improve their utility as sensors as described in U.S. Pat. Nos. 5,405,975 and 5,453,517.

When the fluorophore is a xanthene, the fluorophore is optionally a fluorescein, a rhodol (including any corresponding compounds disclosed in U.S. Pat. Nos. 5,227,487 and 5,442,045), or a rhodamine (including any corresponding compounds in U.S. Pat. Nos. 5,798,276; 5,846,737; and 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Alternatively, the fluorophore is a xanthene that is bound to the thiosulfate moiety either directly or indirectly via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Exemplary fluorophores include xanthene (rhodol, rhodamine, fluorescein and derivatives thereof), coumarin, cyanine, pyrene, oxazine, bimane, borapolyazaindacene, and derivatives thereof. In an exemplary embodiment, the fluorophore is selected from sulfonated xanthenes, fluorinated xanthenes, sulfonated coumarins, fluorinated coumarins and sulfonated cyanines. The choice of the fluorophore attached to the thiosulfate moiety will determine the absorption and fluorescence emission properties of the present compounds and the labeled components. Physical properties of a fluorophore reporter group include spectral characteristics (absorption, emission and Stokes shift), fluorescence intensity, lifetime, polarization and photo-bleaching rate, all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

In one aspect of the invention, the fluorophore has an absorption maximum beyond 480 nm. In a particularly useful embodiment, the fluorophore absorbs at or near 488 nm to 514 nm (particularly suitable for excitation by the output of the argon-ion laser excitation source) or near 546 nm (particularly suitable for excitation by a mercury arc lamp).

Enzymes can be reporter groups for the thiosulfate compounds. Enzymes are desirable reporter groups because amplification of the detectable signal can be obtained resulting in increased assay sensitivity. The enzyme itself usually does not produce a detectable response but functions to break down a substrate when it is contacted by an appropriate substrate such that the converted substrate produces a fluorescent, colored, or luminescent signal. Enzymes amplify the detectable signal because one enzyme can convert multiple molecules of substrate to a detectable signal. This is advantageous where there is a low quantity of target present in the sample or a fluorophore does not exist that will give comparable or stronger signal than the enzyme. However, fluorophores can be preferred as reporter groups because they do not require additional assay steps and thus reduce the overall time required to complete an assay. The enzyme substrate is selected to yield the preferred measurable property, e.g. color, fluorescence, or chemiluminescence. Such substrates are extensively used in the art, many of which are described in the MOLECULAR PROBES HANDBOOK, supra.

A preferred colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants (U.S. Pat. No. 4,384,042) and reduced dihydroxanthenes, including dihydrofluoresceins (U.S. Pat. No. 6,162,931) and dihydrorhodamines including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Another preferred colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912) fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. Preferred fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl beta-D-galactopyranoside, carboxyumbelliferyl beta-D-galactopyranoside and fluorinated coumarin beta-D-galactopyranosides (U.S. Pat. No. 5,830,912).

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In an exemplary embodiment, the enzyme is a member selected from a peroxidase, a phosphatase, a glycosidase, and a luciferase.

In addition to enzymes, haptens such as biotin, desthiobiotin, and digoxigenin are also preferred reporter groups. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

Fluorescent proteins are also examples of reporter groups. Examples of fluorescent proteins include green fluorescent protein (GFP) and the phycobiliproteins and derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger Stokes shift wherein the emission spectra is further shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorophore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in U.S. Pat. No. 5,798,276, or the sulfonated cyanine fluorophores disclosed in U.S. Patent Publication Nos. U.S. 2002-0077487 A1 and U.S. 2002-0064794 A1; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor. In addition, a tandem dye can comprise fluorophores for both the energy donor and acceptor including those combinations disclosed in U.S. Pat. Nos. 5,863,727; 6,358,684; 6,221,606; 6,008,379; 5,945,526; 5,863,727; 5,800,996; 6,335,440; 6,008,373; 6,184,379 and 6,140,494

In an exemplary embodiment, the fluorophore is a fluorescent protein. In a related embodiment, the fluorescent protein is a phycobiliprotein. In a further related embodiment, the phycobiliprotein is selected from a cyanine-labeled phycobiliprotein derivative and a xanthene-labeled phycobiliprotein.

A variety of binding pairs are useful in the current invention. Binding pairs includes a binding pair member and its complementary binding partner. Typically, the binding pair member is capable of specifically interacting with its complementary binding partner. Binding pairs typically contain an area on the surface or in a cavity that specifically binds to, and is complementary with, a particular spatial and polar organization of the corresponding binding pair member or complementary binding partner. In an exemplary embodiment, the specificity of the binding pair is characterized by a dissociation constant (K_{D}) of less than about 10⁻⁶ M.

In another exemplary embodiment, the reporter group is or contains a specific binding pair member. Typically, the reporter group is conjugated to a specific binding pair member and used for formation of the bound pair. In a related embodiment, the presence of the thiosulfate compound having a binding pair member is indicated by the presence of a labeled complementary member of the binding pair.

Exemplary binding pairs are set forth in Table 1.

TABLE 1
Representative Specific Binding Pairs

| antigen | antibody |
|---|---|
| biotin | avidin (or streptavidin or anti-biotin) |
| IgG* | protein A or protein G |
| drug | drug receptor |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| DNA (RNA) | cDNA (cRNA)† |
| hormone | hormone receptor |
| ion | chelator |

*IgG is an immunoglobulin
†cDNA and cRNA are the complementary strands used for hybridization

In another exemplary embodiment, the binding pair member is a protein tag binder and the complementary binding partner is a protein tag. Protein tag binders and protein tags are defined above. Thus in a related embodiment, the reporter group is a protein tag binder and the target analyte (described below) contains protein tag.

In another exemplary embodiment, the reporter group is a phosphorophore (also referred to herein as a phosphorescent dye). Useful phosphorophores are chemical moieties that typically exhibit an absorption maximum beyond 280 nm, and possess attractive spectral properties. Phosphorophores include, without limitation; eosin, erythrosin, acridine, complexes of lanthanides (primarily europium and terbium, emitting orange and green luminescence respectively with lifetimes of the order of 1 ms), porphyrin and phthalocyanine derivatives of the platinum metals, and complexes such as ruthenium bipyridyl and related materials, and deriviatives thereof.

The Z group can comprise a solid support. The solid support is typically substantially insoluble in liquid phases. Solid supports are not limited to a specific type of support. Rather, a large number of supports are available and are known to one of ordinary skill in the art. Thus, useful solid supports include semi-solids, such as aerogels and hydrogels, resins, beads, biochips (including thin film coated biochips), multi-well plates (also referred to as microtitre plates), membranes, conducting and nonconducting metals and magnetic supports. More specific examples of useful solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, ficols, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

A suitable solid phase support can be selected on the basis of desired end use and suitability for various synthetic protocols. For example, where amide bond formation is desirable to attach the thiosulfate moiety or the linker to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), poly(dimethylacrylamide) resin (available from Milligen/Biosearch, California), or PEGA beads (obtained from Polymer Laboratories).

The Z group can comprise a reactive group. The resulting thiosulfate compound can be referred to as a crosslinking reagent due to its ability to form covalent bonds to two different groups.

Reactive groups typically provide an additional locus for attaching the thiosulfate compound to another species to form a conjugate. This attachment is in addition to the attachment possible through use of the thiosulfate group to form a disulfide bond. Exemplary species include biological or non-biological molecules, solid supports, target analytes and the like. The reactive group reacts with a functional group of complementary reactivity at the attachment site of the other species. The reaction leads to the formation of a covalent linkage between the thiosulfate compound and other species.

The reactive group is typically an electrophile or a nucleophile that can generate a covalent linkage. Alternatively, the reactive group is a photoactivatable group, and becomes chemically reactive only after illumination with light of an appropriate wavelength. Typically, the conjugation reaction between the reactive group and the reactive component results in one or more atoms of the reactive group being incorporated into a new linkage attaching the compound or reagents of the invention to the biological or non-biological reactive component. Selected examples of linkages formed between the reactive group and the reactive component are shown in Table 2, where the reaction of an electrophilic group and a nucleophilic group yields a covalent linkage.

TABLE 2
Examples of some routes to useful covalent linkages
with electrophile and nucleophile reactive groups

| Electrophilic Group | Nucleophilic Group | Resulting Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | ami dines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |
| *Activated esters, as understood in the art, generally have the formula —COΩ, where Ω is a good leaving group (e.g. oxysuccinimidyl (—OC₄H₄O₂) oxysulfosuccinimidyl (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, | | |
| # or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl). | | |

**Acyl azides can also rearrange to isocyanates

The selection of a covalent linkage to attach the reactive component typically depends on the chemically reactive group on the reactive component to be conjugated (hereinafter referred to as the “reactive component functional group”). Exemplary reactive component functional groups typically include, but are not limited to, amines, thiols, alcohols, phenols, aldehydes, ketones, phosphates, imidazoles, hydrazines, hydroxylamines, disubstituted amines, halides, epoxides, sulfonate esters, purines, pyrimidines, carboxylic acids, or a combination of these groups. A single type of reactive component functional group may be available on the reactive component (typical for polysaccharides), or a variety of sites may occur (e.g. amines, thiols, alcohols, phenols), as is typical for proteins. Although some selectivity can be obtained by careful control of the reaction conditions, selectivity of labeling is best obtained by selection of an appropriate reactive group.

In an exemplary embodiment, the reactive groups react with an amine, a thiol or an alcohol. In another exemplary embodiment, the reactive group is an acrylamide, an activated ester of a carboxylic acid, an acyl azide, an acyl nitrile, an aldehyde, an alkyl halide, an amine, an anhydride, an aniline, an aryl halide, an azide, an aziridine, a boronate, a carboxylic acid, a diazoalkane, a haloacetamide, a halotriazine, a hydrazine, an imido ester, an isocyanate, an isothiocyanate, a maleimide, a phosphoramidite, a sulfonyl halide, or a thiol group.

Where the reactive group is an activated ester of a carboxylic acid, the resulting crosslinking reagent is particularly useful for preparing conjugates wherein the reactive component is a protein or nucleic acid, e.g., nucleotides and oligonucleotides, or haptens. Where the reactive group is a maleimide or haloacetamide the resulting crosslinking reagent is particularly useful for conjugation to thiol-containing substances. Where the reactive group is a hydrazide, the resulting crosslinking reagent is particularly useful for conjugation to periodate-oxidized carbohydrates and glycoproteins, and in addition is an aldehyde-fixable polar tracer for cell microinjection. Where the reactive group is a silyl halide, the resulting crosslinking reagent is particularly useful for conjugation to silica surfaces, particularly where the silica surface is incorporated into a fiber optic probe subsequently used for remote ion detection or quantitation or forms the substrate of a microarray or biochip.

In another exemplary embodiment, the reactive group is a succinimidyl ester of a carboxylic acid, a haloacetamide, a hydrazine, an isothiocyanate, a maleimide group, an aliphatic amine, a silyl halide, or a psoralen. In a related embodiment, the reactive group is a succinimidyl ester of a carboxylic acid, a maleimide, an iodoacetamide, or a silyl halide.

The preparation of conjugates using reactive groups is well documented, Haugland, MOLECULAR PROBES, INC. HANDBOOK OF FLUORESCENT PROBES AND RESEARCH CHEMICALS, 1996 and Brinkley, Bioconjugate Chem., 3: 2 (1992). The compounds can be dissolved in water or a water-miscible such as a lower alcohol, dimethylformamide (DMF), dimethylsulfoxide (DMSO), acetone, acetonitrile, tetrahydrofuran (THF), or dioxane prior to conjugation. Conjugates typically result from mixing appropriate reactive compounds and the reactive component to be conjugated in a suitable solvent in which both are soluble, using methods well known in the art, followed by separation of the conjugate from any unreacted reactive component and by-products. The present crosslinking reagents are typically combined with the reactive component under appropriate conditions (e.g., concentration, stoichiometry, pH, and temperature) to effect the desired reaction. These factors are generally well known in the art of forming bioconjugates (Haugland et al. “Coupling of Antibodies with Biotin” THE PROTEIN PROTOCOLS HANDBOOK, J. M. Walker, ed., Humana Press, (1996); Haugland “Coupling of Monoclonal Antibodies with Fluorophores” METHODS IN MOLECULAR BIOLOGY, VOL. 45: MONOCLONAL ANTIBODY PROTOCOLS, W. C. Davis, Ed. (1995)). For those crosslinking reagents that are photoactivated, conjugation requires illumination of the reaction mixture to activate the crosslinking reagent. The conjugate may be used in solution or lyophilized and stored for later use.

The crosslinking reagents can be used to label reactive sites such as occur at the surface of cells, in cell membranes or in intracellular compartments such as organelles, or in the cell's cytoplasm, or to derivative low molecular weight compounds for their analysis by capillary zone electrophoresis (CZE), HPLC or other separation techniques. Certain reactive groups allow the retention of the crosslinking reagent in cells or organelles by reacting with cellular materials. In particular, haloalkyl- or halomethylbenzamide-substituted fluorinated fluorophores are used to react selectively with intracellular reactive components such as glutathione, or to retain the dye compounds within cells or within selected organelles where the dye compound is localized therein, according to methods previously described (U.S. Pat. Nos. 5,362,628; 5,576,424 (in cells); U.S. Pat. No. 5,459,268; 5,686,261 (in mitochondria). Polyfluoroaryl-substituted dye compounds are similarly retained in cells, in part by covalent attachment. The crosslinking reagents may be used to localize staining in a part of the sample, e.g., where the localization of the corresponding functional group is indicative of a characteristic of the sample; or to retain the dye in a specific portion of the sample for extended periods of time, e.g., to follow the stained portion of the sample through a period of time or sequence of events.

The thiosulfate compounds can optionally further comprise a linker L. When the linker is present, the thiosulfate compound would have the structure Z-L-S—SO₃H or its conjugate base Z-L-S—SO₃⁻. The linker can generally be any linker. Examples of linkers include a branched- or straight-chain, saturated or unsaturated chain of atoms, e.g., substituted or unsubstituted alkyl, and substituted or unsubstituted heteroalkyl. Examples of linkers include substituted or unsubstituted polyalkylene, arylene, alkylarylene, arylenealkyl, or arylthio groups.

Thus, the thiosulfate group may be directly attached to the Z group (without a linker), or attached through a linker containing one or more atoms and a series of stable bonds. The linker typically incorporates 1 to about 20, more preferably 1 to about 15, non-hydrogen atoms selected from the group consisting of C, N, O, S, P, and combinations thereof. Specific numbers of non-hydrogen atoms in the linker includes 1, 2, 3, 4, 5, 6, 7, 8, 9, 10, 11, 12, 13, 14, 15, 16, 17, 18, 19, 20, and ranges between any two of these values. In addition, the linker can incorporate a platinum atom, such as described in U.S. Pat. No. 5,714,327.

The linker may contain any combination of chemical bonds, including single, double, triple or aromatic carbon-carbon bonds, as well as carbon-nitrogen bonds, nitrogen-nitrogen bonds, carbon-oxygen bonds, carbon-sulfur bonds, phosphorus-oxygen bonds, phosphorus-nitrogen bonds, and nitrogen-platinum bonds. Exemplary components of the linker include ether, thioether, thiourea, amine, ester, carboxamide, sulfonamide, hydrazide bonds and aromatic or heteroaromatic bonds.

An exemplary linker is a combination of single carbon-carbon bonds and carboxamide, sulfonamide or thioether bonds. The bonds of the linker typically result in the following moieties that can be found in the linker: ether, thioether, carboxamide, thiourea, sulfonamide, urea, urethane, hydrazine, alkyl, aryl, heteroaryl, alkoxy, cycloalkyl and amine moieties.

Any combination of linkers may be used in the thiosulfate compounds. An exemplary thiosulfate compound, when attached to more than one thiosulfate group will have one or two linkers attached that may be the same or different. The linker may also be substituted to alter the physical properties of the present compounds, such as solubility and spectral properties of the thiosulfate compound.

In an exemplary embodiment, the linker is selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. In a related embodiment, the linker is selected from substituted or unsubstituted (C₁-C₁₅) alkylene, and substituted or unsubstituted 2-10-member heteroalkylene.

In another exemplary embodiment, the linker further contains a reactive group capable of forming a covalent bond to another functional group. Useful reactive groups include, for example: (a) carboxyl groups and derivatives thereof including, but not limited to activated esters, e.g., N-hydroxysuccinimide esters, N-hydroxybenztriazole esters, acid halides, acyl imidazoles, thioesters, p-nitrophenyl esters, alkyl, alkenyl, alkynyl and aromatic esters, activating groups used in peptide synthesis and acid halides; (b) hydroxyl groups, which can be converted to esters, sulfonates, phosphoramidites, ethers, aldehydes, etc.; (c) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups, which are capable of participating in Diels-Alder reactions such as, for example, maleimido groups; (e) aldehyde or ketone groups, allowing derivatization via formation of carbonyl derivatives, e.g., imines, hydrazones, semicarbazones or oximes, or via such mechanisms as Grignard addition or alkyllithium addition; (f) sulfonyl halide groups for reaction with amines, for example, to form sulfonamides; (g) thiol groups, which can be converted to disulfides or reacted with acyl halides, for example, although thiol groups would react with the thiosulfate group to form a disulfide bond; (h) amine or sulfhydryl groups, which can be, for example, acylated, alkylated or oxidized; (i) alkenes, which can undergo, for example, cycloadditions, acylation, Michael addition, etc; (j) epoxides, which can react with, for example, amines and hydroxyl compounds; and (k) phosphoramidites and other standard functional groups useful in nucleic acid synthesis. Further examples of useful reactive groups are discussed below in the context of reactive groups, which are equally applicable here.

Linkers can be chosen to provide a desired distance between the thiosulfate group and the Z group. Linkers are typically substantially chemically inert so as to avoid undesired intramolecular or intermolecular cross reactions. Linkers can be hydrophilic (e.g., tetraethylene glycol, hexaethylene glycol, polyethylene glycol) or they can be hydrophobic (e.g., hexylene, decylene, etc.). In an exemplary embodiment, the linker is selected from substituted or unsubstituted C₁-C₃₀ alkyl groups, polyols, polyethers (e.g., poly(ethylene glycol)), polyamines, polyamino acids, polysaccharides and combinations thereof.

In an exemplary embodiment, the linker has the formula:

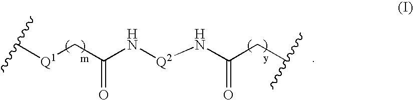

In Formula (I), m and y are individually selected from the integers 0 to 10. Q¹ is selected from a bond, —O—, —S—, —NH—, substituted or unsubstituted alkylene, and substituted or unsubstituted heteroalkylene. Q² is selected from substituted or unsubstituted alkylene and substituted or unsubstituted heteroalkylene. In a related embodiment, Q¹ is selected from a bond, —O—, —S—, —NH—, substituted or unsubstituted (C₁-C₁₀) alkylene, and substituted or unsubstituted 2-10-member heteroalkylene. In another related embodiment, Q¹ is selected from a bond, —O—, —S—, —NH—, substituted or unsubstituted (C₁-C₅) alkylene, and substituted or unsubstituted 2-5-member heteroalkylene. Q² may be selected from substituted or unsubstituted (C₁-C₁₀) alkylene, and substituted or unsubstituted 2-10-member heteroalkylene. In a related embodiment, Q² is selected from substituted or unsubstituted (C₁-C₅) alkylene, and substituted or unsubstituted 2-5-member heteroalkylene. In another exemplary embodiment, m and y are individually selected from the integers 0 to 5. In a related embodiment, Q¹ is —O—, Q² is an unsubstituted (C₁-C₅) alkylene, and m and y are 1.

In another exemplary embodiment, the linker has the formula:

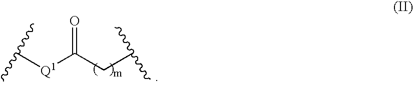

In Formula (II), m is selected from the integers 0 to 10, and Q¹ is selected from —O—, —S—, and —NH—. In a related embodiment, m is individually selected from the integers 0 to 5.

The term “specifically forms” or “specifically contacts” refers to the ability of the thiosulfate group to form a disulfide bond by reacting with a thiol rather than with a non-thiol nucleophile that may be present. For example, a protein typically contains a variety of non-thiol nucleophiles that are capable of forming covalent bonds with selected reactive species, such as carboxylic acids, amines, and amides. In an exemplary embodiment, the thiosulfate compounds will only form a disulfide bond with a thiol of a protein rather than a covalent bond with a non-thiol nucleophile such as a carboxylic acid, amine, or amide of the protein. In addition, nucleic acids, including nucleic acid analogs, will also typically contain a variety of non-thiol nucleophiles, such as phosphoesters, amines, and amides. In an exemplary embodiment, the thiosulfate compounds will form a disulfide bond with a thiol that has been added to a nucleic acid, rather than form a covalent bond with a carboxylic acid, amine, or amide group of the nucleic acid.

The specificity of the thiosulfate group to react with a thiol group can be measured using a wide variety of methods. For example, a population of thiosulfate compounds can be combined with a population of target analytes having a thiol group in the presence of one or more non-thiol nucleophiles. Typically, the target analyte will contain at least one non-thiol nucleophile. After combining the thiosulfate compounds with the target analytes, at least one disulfide bond is allowed to form between the thiosulfate moiety and the thiol moiety. After formation of the at least one disulfide bond, the total amount of target analyte covalently bound to the thiosulfate compound is determined, typically using a technique that includes separation of the conjugate from the thiosulfate compound. A target analyte covalently bound to the thiosulfate compound is also referred to herein as target analyte conjugate. The total amount of target analyte conjugate will include those conjugates having non-disulfide covalent linkages between the thiosulfate moiety and non-thiol nucleophiles of the target analyte. After determining the total amount of target analyte conjugate, the target analyte conjugates are contacted with a reducing agent thereby reducing the at least one disulfide bond. The amount of remaining target analyte conjugate is then determined, thereby determining the portion of the total amount of target analyte conjugates having a disulfide linkage.

Methods of Preparation

The thiosulfate compounds are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention, it is not intended to define the scope of reactions or reaction sequences that are useful in preparing the compounds of the present invention.

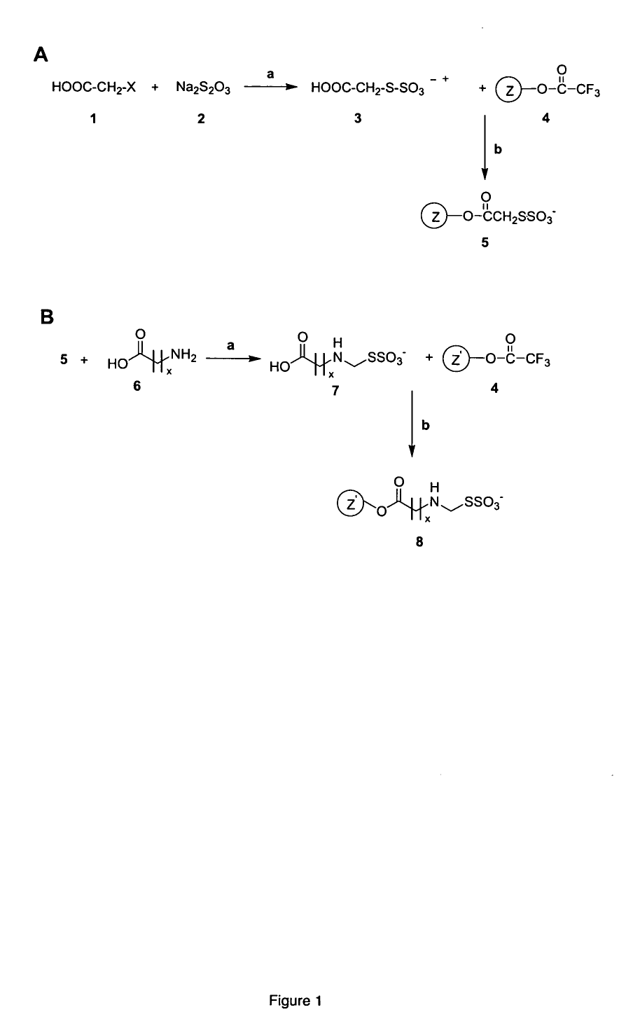

FIG. 1A shows a synthetic method to prepare a thiosulfate compound. A haloacetic acid 1 is added to sodium thiosulfate 2 to form the carboxymethyl thiosulfate, 3 (reaction a), which is then combined with the trifluoroacetate species 4 to yield the thiosulfate compound 5 (reaction b).

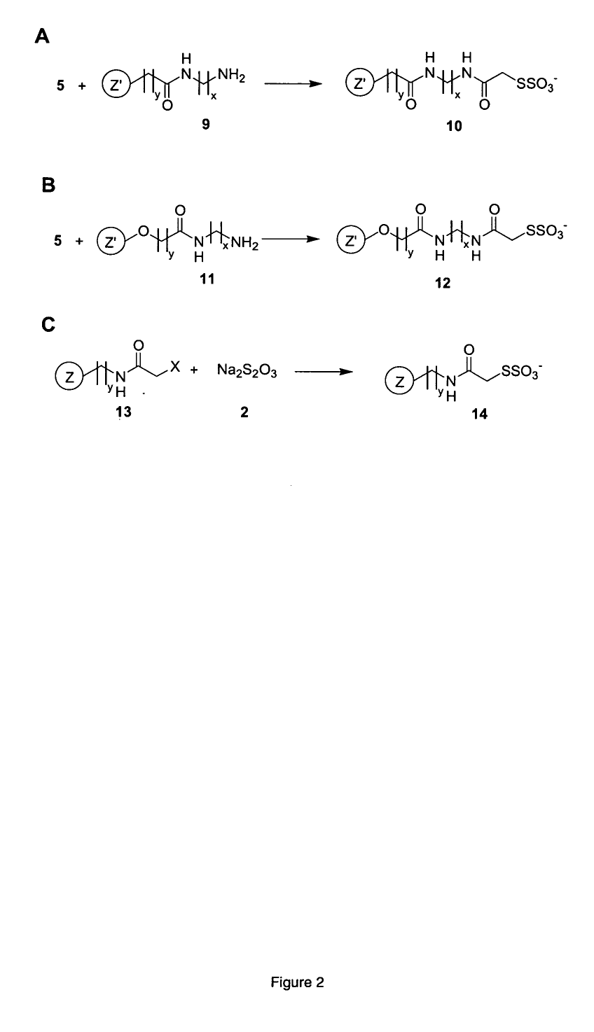

Thiosulfate compound 5 can be further reacted to form other thiosulfate compounds, as shown in FIGS. 2A-C. Subscripts x and y in the Figures are integers individually selected from 0 to 10.

In FIG. 1B, thiosulfate compound 5 is reacted with the aminoalkanoic acid 6 to yield the thiosulfate compound 7 having a carboxylic acid reactive group, which can serve as a crosslinking reagent. Compound 7 is then reacted with compound 4 to yield the thiosulfate compound 8.

In FIG. 2A, compound 5 is combined with the amine species 9 to yield the thiosulfate compound 10.

In FIG. 2B, compound 5 is combined with the amine species 11 to yield the thiosulfate compound 12.

In FIG. 2C, compound 2 is combined with the haloacetamido species 13 to yield thiosulfate compound 14.

The thiosulfate compounds 5, 7, 10, 12 and 14 can be reacted with a thiol moiety to from a disulfide bond. Illustrative conditions are described in Examples 10 and 11.

Other moieties are optionally present during these synthetic steps. They may either be protected with a protecting group or resistant to the steps required for synthesis of the thiosulfate compounds. Exemplary moieties include alkyl, carboxyalkyl, chloro, bromo, iodo, alkoxy and hydroxy groups. Hydroxy moieties may also be formed during cleavage of alkoxy groups present in the starting material.

Embodiments of the invention also includes the synthetic methods and intermediates described above. A method of preparing a thiosulfate compound can comprise contacting a haloacetic acid and a thiosulfate salt (such as ammonium thiosulfate, potassium thiosulfate, barium thiosulfate, or calcium thiosulfate) to prepare a carboxymethyl thiosulfate; and contacting the carboxymethyl thiosulfate with Z-OC(O)CF₃ to prepare a Z-OC(O)CH₂S₂O₃⁻thiosulfate compound, where Z is the component to which a thiosulfate group is desired to be covalently attached. The intermediate is the carboxymethyl thiosulfate compound. The method can further comprise one or more purification steps of the intermediate, the product, or both.

An alternate method of preparing a thiosulfate compound can comprise contacting a haloacetamido species Z-(CH₂)_{y}NHC(O)CH₂X and a thiosulfate salt (such as ammonium thiosulfate, potassium thiosulfate, barium thiosulfate, or calcium thiosulfate) to prepare the thiosulfate compound Z-(CH₂)_{y}NHC(O)CH₂S₂O₃—. In the chemical structures, X is a halogen such as chlorine, bromine, or iodine, and Y is a positive integer such as 0, 1, 2, 3, 4, 5, 6, 7, 8, 9, 10, 11, 12, and so on. Y can be a positive integer or a positive non-zero integer.

Additional synthetic methods and intermediates are also described above. For example, compound 5 and 6 can be contacted to prepare compound 7. Compound 7 can be contacted with compound 4 to prepare thiosulfate compound 8. Compound 5 can be contacted with compound 9 to prepare thiosulfate compound 10. Compound 5 can be contacted with compound 11 to prepare thiosulfate compound 12.

Methods of Use

The present invention also provides methods of using the compounds described herein for a wide variety of chemical, biological and biochemical applications.

One embodiment of the invention includes methods for preparing a disulfide bond. The method includes contacting one of the above described thiosulfate compounds (Z-S-SO₃H, Z-S—SO₃⁻, Z-L-S—SO₃H, or Z-L-S—SO₃⁻) with a target comprising a thiol (—SH) group. The thiosulfate compound and target are allowed to contact under conditions suitable for formation of a disulfide bond by reaction of the thiosulfate group and the thiol group. The method can further comprise detecting the presence of the disulfide bond linked compound and target. Detection of the disulfide bond can be used to assay the presence or absence of the target comprising the thiol in a sample. The method can further comprise regenerating the thiol by use of a reducing agent. The reducing agent can generally be any reducing agent, such as a thiol or a phosphine.

An alternative embodiment of the invention provides methods for preparing an intramolecular disulfide bond. The method comprises providing a compound containing both a thiosulfate group and a thiol group. The compound is maintained under conditions suitable for intramolecular reaction of the two groups, resulting in the formation of an intramolecular disulfide bond. The thiol group can initially be present as a protected thiol group, which once deprotected, reacts with the thiosulfate group.

An additional embodiment of the invention provides methods of forming an immobilized target, where the target contains a thiol group. The method comprises contacting a sample containing the target with a solid support covalently bonded to a thiosulfate moiety. The sample and the solid support are contacted under conditions suitable for the formation of a disulfide bond by reaction of the thiosulfate group and the thiol group. The method results in immobilization of the target on the solid support. The method can further comprise detecting the immobilized target. The method can further comprise washing (e.g., rinsing the solid support to remove components of the sample other than the immobilized target) or otherwise removing the sample from the immobilized target (e.g., by physically removing the solid support from the sample). The method can further comprise releasing the immobilized target from the solid support by use of a reducing agent such as a thiol or a phosphine. These methods can be used to capture a thiol-containing target from a sample, or viewed differently, to remove an undesired thiol-containing target or targets from a sample.

An alternative embodiment of the invention provides a method of forming an immobilized target, where the target contains a thiosulfate group. The method comprises contacting a sample containing the target with a solid support covalently bonded to a thiol moiety. The sample and the solid support are contacted under conditions suitable for the formation of a disulfide bond by reaction of the thiosulfate group and the thiol group. The method results in immobilization of the target on the solid support. The method can further comprise detecting the immobilized target. The method can further comprise washing (e.g., rinsing the solid support to remove components of the sample other than the immobilized target) or otherwise removing the sample from the immobilized target (e.g., by physically removing the solid support from the sample). The method can further comprise releasing the immobilized target from the solid support by use of a reducing agent such as a thiol or a phosphine.

An alternative embodiment of the invention provides a method of forming a crosslinked compound.

A wide variety of targets can be used in the above described methods. Targets include any microorganism, compound or molecule of interest for which a diagnostic test is desired. A target can be, for example, a protein, peptide, carbohydrate, liposome, fatty acid, phospholipid, triacylglycerol, polysaccharide, glycoprotein, hormone, receptor, antigen, antibody, cells (such as pathogenic microorganism), viruses, substrate, pharmaceutical, metabolite, narcotic, poison, transition state analog, cofactor, inhibitor, dye, nutrient, growth factor, antibodies, enzymes, protein tags, complementary binding partners and derivatives and combinations thereof, without limitation. A target may be a single component or a plurality of components in a given sample.

In an exemplary embodiment, the target is selected from a polypeptide, a protein, a polysaccharide, a nucleic acid, a carbohydrate, a hapten, a psoralen, a drug, a toxin, a hormone, a lipid, a lipid assembly, a synthetic polymer, a polymeric microparticle, a biological cell, a virus and combinations thereof.

A wide variety of reducing agents are useful in reducing the disulfide bonds in the methods of the current invention, including thiol reducing agents (including bismercaptans), phosphine reducing agents, phosphite reducing agents, zinc metal powder reducing agents and sodium hydrosulfite reducing agents. More specific examples of useful reducing agents include 2-mercaptoethanol, 2-mercaptoethylamine.HCl, dithiothreitol (DTT), dithioerythritol, Ellman's Reagent, TCEP, N-ethylmaleimide, tributyl phosphine, triphenyl phosphine, and triethyl phosphite.

Rinsing the solid support typically functions to remove residual, excess, or unbound materials from the solid support other than the immobilized target. Any appropriate solution or series or solutions may be used to rinse the solid support. Exemplary solvents useful in the present invention include both polar and non-polar solvents. Thus, any appropriate organic solvent or aqueous solution is useful in the methods of the current invention.

Solutions of the compounds of the invention can be prepared according to methods generally known in the art. The thiosulfate compounds are generally soluble in water and aqueous solutions having a pH greater than or equal to about 6. Stock solutions of pure thiosulfate compounds, however, may be dissolved in organic solvent before diluting into aqueous solution or buffer. Exemplary organic solvents are aprotic polar solvents such as DMSO, DMF, N-methylpyrrolidone, acetone, acetonitrile, dioxane, tetrahydrofuran and other nonhydroxylic, completely water-miscible solvents. The concentration of thiosulfate compound to be used is dependent upon the experimental conditions and the desired results, and routine optimization of experimental conditions can be performed to determine the best concentration of thiosulfate compound to be used in a given application. The concentration of thiosulfate compounds typically ranges from millimolar to micromolar. The optimal concentration for the thiosulfate compounds can be determined by systematic variation in thiosulfate compound concentration until satisfactory results are accomplished. The starting ranges are readily determined from methods known in the art for use of similar compounds under comparable conditions for the desired response.

For those compounds of the present invention that are substituted by lipophilic moieties, the thiosulfate compounds are optionally introduced into living cells by passive permeation through the cellular membranes. Less cell-permeant embodiments of the invention are optionally introduced into cells by pressure microinjection methods, scrape loading techniques (short mechanical disruption of the plasma membrane where the plasma membrane is peeled away from the cytoplasm, the dye is perfused through the sample and the plasma membrane is reassembled), patch clamp methods (where an opening is maintained in the plasma membrane for long periods) or phagocytosis. Any other treatment that will permeabilize the plasma membrane, such as electroporation, shock treatments or high extracellular ATP may be used to introduce the thiosulfate compounds into the cellular cytoplasm.

Any suitable method of detection is useful in detecting labeled targets or immobilized targets, such as those based on fluorescence, phosphorescence and/or radiation. In an exemplary embodiment, detection is achieved by illuminating the labeled target or immobilized target at a wavelength selected to elicit a detectable optical response, where the target has been labeled with a fluorescent or otherwise detectable group.

A detectable optical response means a change in, or occurrence of, a parameter in a test system that is capable of being perceived, either by direct observation or instrumentally. Such detectable responses include the change in, or appearance of, color, fluorescence, reflectance, chemiluminescence, light polarization, light scattering, x-ray scattering, and radioactivity. Typically, the detectable response is a change in fluorescence, such as a change in the intensity, excitation or emission wavelength distribution of fluorescence, fluorescence lifetime, fluorescence polarization, or a combination thereof. The detectable optical response may occur throughout the sample or in a localized portion of the sample. The presence or absence of the optical response after the elapsed time is indicative of one or more characteristic of the sample. Comparison of the amount of thiosulfate compound with a standard or expected response can be used to determine whether and to what degree sample possesses a target analyte containing a thiol moiety.

The thiosulfate compounds and conjugates are useful as coloring agents, tracers for detecting the flow of fluids such as in angiography, and tracing of fluid flow through gap junctions of neurons according to procedures known in the art for other dyes. The thiosulfate compounds are also useful in assays as haptens.

The compounds of the invention are also of use in the numerous fluorescence polarization-based assays.

In those embodiments in which a thiosulfate moiety is covalently bound to a carrier molecule that is a chelator of calcium, sodium, magnesium, potassium, or other biologically important metal ion, the thiosulfate compound functions as an indicator of the ion. The compound can optionally be further conjugated to a biological or plastic polymer according to methods known in the art; e.g., using fluorinated analogs of the compounds described in U.S. Pat. Nos. 5,453,517; and 5,405,975. Alternatively, the thiosulfate compound can act as a pH indicator at pH values within about 1.5 pH units of the individual dye's pKa. Typically the detectable optical response of the ion indicators is a change in fluorescence of the ion chelator.

Illumination

Where the methods of the present invention employ a reporter group that is a dye, the reporter group may, at any time after or during an assay, be illuminated with a wavelength of light that results in a detectable optical response, and observed with a means for detecting the optical response. While the dye compounds are detectable colorimetrically, using ambient light, typically the dye compounds are detected by the fluorescence properties of the parent fluorophore. Upon illumination, such as by an ultraviolet or visible wavelength emission lamp, an arc lamp, a laser, or even sunlight or ordinary room light, the dye compounds, including dye compounds bound to the complementary specific binding pair member, display intense visible absorption as well as fluorescence emission. Selected equipment that is useful for illuminating the dye-conjugates of the invention includes, but is not limited to, hand-held ultraviolet lamps, mercury arc lamps, xenon lamps, argon lasers, laser diodes, and YAG lasers. These illumination sources are optionally integrated into laser-based scanners, fluorescence-based microplate readers, standard or mini fluorometers, or chromatographic detectors. This colorimetric absorbance or fluorescence emission is optionally detected by visual inspection, or by use of any of the following devices: CCD cameras, video cameras, photographic film, laser-based scanning devices, fluorometers, photodiodes, quantum counters, epifluorescence microscopes, scanning microscopes, flow cytometers, fluorescence-based microplate readers, or by a unit for amplifying the signal such as photomultiplier tubes. Where a flow cytometer, a fluorescence microscope or a fluorometer is employed, the instrument is optionally used to distinguish and discriminate between the reporter group and a second fluorophore with detectably different optical properties, typically by distinguishing the fluorescence response of the reporter group from that of the second fluorophore. Where a sample is examined using a flow cytometer, examination of the sample optionally includes isolation of particles within the sample based on the fluorescence response of the reporter group by using a sorting device.

Sample Preparation

The end user will determine the choice of the sample and the way in which the sample is prepared. The sample includes, without limitation, any biological derived material or aqueous solution that is thought to contain a thiol moiety or target analyte containing a thiol moiety.

The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells and eukaryotic cells that include primary cultures and immortalized cell lines. Eukaryotic cells include without limitation ovary cells, epithelial cells, circulating immune cells, β cells, hepatocytes, and neurons.

Kits

In another aspect, the present invention provides kits that include a thiosulfate compound. The kit will generally also include instructions for using the thiosulfate compound in one or more methods.

In an exemplary embodiment, the kit includes a thiosulfate compound containing a reporter group, and instructions for labeling a target with the compound.

The instructions can describe contacting a target having a thiol group with the thiosulfate compound, and allowing the thiosulfate group to react with the thiol group to form a disulfide bond, thereby forming the labeled target.

The instructions can further specify, after forming the labeled target, detecting the presence of the labeled target, thereby detecting the target comprising the thiol group in a sample. In another related embodiment, the instructions further specify, after forming the labeled target, regenerating the thiol group using a reducing agent. In another related embodiment, the reducing agent is a selected from a thiol compound and a phosphine compound.

In another exemplary embodiment, the kit includes a solid support covalently bonded to a thiosulfate group, and instructions for immobilizing a target in a sample to the solid support.

In a related embodiment, the instructions specify combining the sample with the solid support covalently bonded to a thiosulfate group, and allowing the thiosulfate group to react with the thiol group of the target to form a disulfide bond, thereby forming an immobilized target.

In another related embodiment, the instructions further specify, after forming the immobilized target, rinsing the solid support to remove components of the sample other than the target. In another related embodiment, the instructions further specify, after forming the immobilized target, detecting the immobilized target. In another related embodiment, the instructions further specify, after forming the immobilized target, releasing the immobilized target using a reducing agent. In another related embodiment, the reducing agent is a member selected from a thiol compound and a phosphine compound.

In another exemplary embodiment, the kit includes a crosslinking reagent having a thiosulfate group and a reactive group each covalently bonded to a linker, and instructions for crosslinking a first target and a second target. In a related embodiment the kit further contains a solid support comprising a thiol moiety.

In a related embodiment, the instructions specify contacting the first target with the reactive group of the crosslinking reagent thereby forming a labeled target covalently bonded to a thiosulfate moiety through a linker. The instructions further specify allowing the thiosulfate moiety of the labeled target to react with the thiol moiety of the solid support to form a disulfide bond thereby forming an immobilized target.

In another related embodiment, the instructions further specify, after forming the immobilized target, rinsing the solid support to remove components of the sample other than the target. In another related embodiment, the instructions further specify, after forming the immobilized target, detecting the immobilized target. In another related embodiment, the instructions further specify, after forming the immobilized target, releasing the immobilized target using a reducing agent. In another related embodiment, the reducing agent is a member selected from a thiol compound and a phosphine compound.

Methods of detecting immobilized target and labeled target are presented in detail above and are equally applicable to the kits.

Those skilled in the art will appreciate that a wide variety of additional kits and kit components can be prepared according to the present invention, depending upon the intended user of the kit, and the particular needs of the user.

The following examples are included to demonstrate preferred embodiments of the invention. It should be appreciated by those of skill in the art that the techniques disclosed in the examples which follow represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Preparation of a Tetrafluorophenyl Thiosulfate Compound

To a solution of bromoacetic acid (20 g, 0.144 mol) in 100 mL of methanol was added a solution of sodium thiosulfate pentahydrate (40 g, 0.161 mol) in 150 mL of water and the mixture was stirred at room temperature for 2 hours. It was then concentrated in vacuo and the resulting residue was suspended in 50 mL of methanol. It was filtered to remove NaBr and the filtrate was concentrated in vacuo. The resulting residue was suspended with acetone (150 mL) and heated under reflux for 15 minutes. It was filtered while warm and the filter cake was heated under reflux with fresh acetone for 15 minutes. This extraction with acetone was done two more times. The combined acetone solution was concentrated to a volume of about 30 mL. The resulting colorless solid was collected by filtration to give 10 g of carboxymethyl thiosulfate, sodium salt. To a solution of this carboxymethyl thiosulfate, sodium salt (1.95 g, 0.010 mol) in 15 mL of DMF was added N,N-diisopropylethylamine (2.2 mL, 0.013 mol), followed by addition of 2,3,5,6-tetrafluorophenyl trifluoroacetate (3.4 g, 0.013 mol). After the reaction mixture was stirred at room temperature for 3 hours, it was added dropwise into about 250 mL of ether and stirred for 1 hour. The ether solution was carefully decanted and a second volume (250 mL) of ether was added to the residue. After being stirred for 1 hour, the resulting solid was collected by filtration and washed with ether (3×15 mL) to give 2.35 g of a colorless powdery solid. TLC: R_{f}=0.28 (silica gel, 30% methanol in chloroform ¹HNMR (D₂O), δ 7.28-7.15 (m, 1H, ArH), 4.19 (s, 2H, CH₂), 3.66-3.50 (m, 2H, 2×CH), 1.23 (t, 3H, CH₃), 1.20 (t, 6H, 2×CH₃). The structure of the resulting tetrafluorophenyl thiosulfate compound is provided below in Formula (EI).

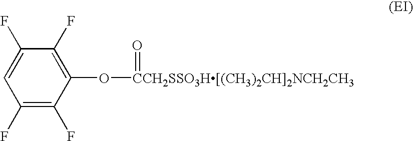

### Example 2

### Preparation of a Tetrafluorophenyl Thiosulfate Compound

To a solution of 6-aminohexanoic acid (0.15 g, 1.13 mmol) in 7 mL of water was added N,N-diisopropylethylamine (320 μL, 1.85 mmol) followed by addition of 2,3,5,6-tetrafluorophenoxycarbonylmethyl thiosulfate (0.51 g, 1.15 mmol) and the mixture was stirred at room temperature for 1 hour. The whole reaction mixture was poured into ether (100 mL) and the resulting precipitate was collected by filtration. After being dried in vacuo, it was dissolved in 5 mL of DMF. To this solution was added N,N-diisopropylethylamine (410 μL, 2.35 mmol) followed by addition of 2,3,5,6-tetrafluorophenyl trifluoroacetate (0.60 g, 2.35 mmol) and the mixture was stirred at room temperature for 4 hours. The reaction mixture was poured into ether (100 mL) and the resulting precipitate was collected by filtration. This crude product was purified by LH-20 column (Sephadex) eluting with water. It was then converted to sodium form by treating with Dowex 50WX (sodium form) to give 460 mg of a product as a white solid. TLC (silica gel): Rf=0.45 (30% methanol in chloroform), ¹HNMR (D₂O): δ 1.32-1.37 (m, 2H, CH₂), 1.44-1.49 (m, 2H, CH₂), 1.62-1.70 (m, 2H, CH₂), 2.68 (t, 2H, CH₂), 3.65 (s, 2H, CH₂), 7.10-7.25 (m, 1H, ArH). The structure of the resulting thiosulfate conjugate is provided below in Formula (E2).

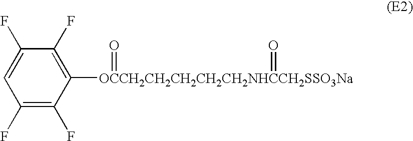

### Example 3

### Preparation of Boradiazaindacene Thiosulfate

To a solution of 4,4-difluoro-5,7-dimethyl-4-bora-3a,4a-diaza-s-indacene-3-propionylethylenediamine, hydrochloride (358 mg, 1.00 mmol) and N,N-diisopropylethylamine (200 μL, 1.16 mmol) in 50 mL of methanol was added a solution of 2,3,5,6-tetrafluorophenoxycarbonylmethyl thiosulfate, N,N-diisopropylethylamine salt (530 mg, 1.16 mmol) in 4 mL of dry DMF. After being stirred at room temperature for 2 hours, the reaction mixture was concentrated in vacuo. The crude product was purified by column chromatography on silica gel eluting with 10% methanol in chloroform. It was then converted to sodium form by treating with Dowex 50 WX (sodium form) to give 421 mg of an orange solid. TLC: Rf=0.34 (silica gel, 25% methanol in chloroform). Absorption maximum: 504 nm in methanol; emission maximum: 510 nm in methanol. The structure of the resulting boradiazaindacene is provided below in Formula (E3). 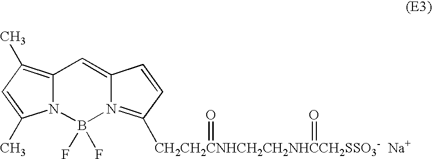
sulfate

### Example 4

### Preparation of a Boradiazaindacene Thiosulfate

To a solution of 5-(((4-(4,4-difluoro-5-(2-thienyl)-4-bora-3a,4a-diaza-s-indacene-3-yl)phenoxy)acetyl)amino)pentylamine, hydrochloride was added 2,3,5,6-tetrafluorophenoxycarbonylmethyl thiosulfate, N,N-diisopropylethylamine salt to give a dark purple solid. TLC: R_{f}=0.56 (silica gel, 25% methanol in chloroform). Absorption maximum: 589 nm in methanol; emission maximum: 618 nm in methanol. The structure of the resulting boradiazaindacene is provided below in Formula (E4).

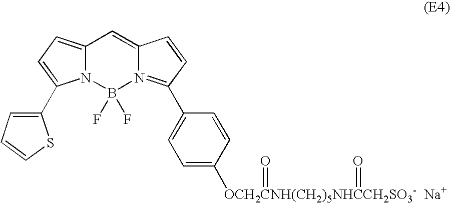

### Example 5

### Preparation of a Boradiazaindacene Thiosulfate

To a solution of 2,3,5,6-tetrafluorophenoxycarbonylmethyl thiosulfate, N,N-diisopropylethylamine salt was added the corresponding borapolyazaindacene TMR amine analog to give a dark red solid. TLC: R_{f}=0.38 (silica gel, 25% methanol in chloroform). Absorption maximum: 544 nm in methanol; emission maximum: 571 nm in methanol. The structure of the resulting boradiazaindacene is provided below in Formula (E5).

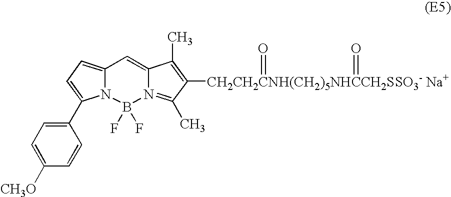

### Example 6

### Preparation of a Boradiazaindacene Thiosulfate

To a solution of 2,3,5,6-tetrafluorophenoxycarbonylmethyl thiosulfate, N,N-diisopropylethylamine salt was added the corresponding borapolyazaindacene amine analog to give a dark blue solid. TLC: R_{f}=0.62 (silica gel, 25% methanol in chloroform). Absorption maximum: 625 nm in methanol; emission maximum: 640 nm in methanol. The structure of the resulting boradiazaindacene is provided below in Formula (E6).

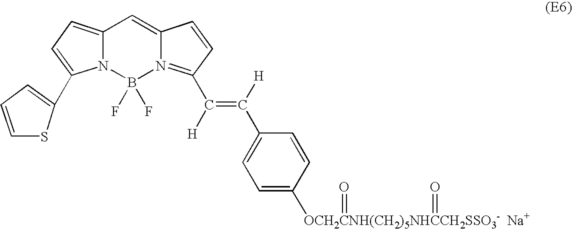

### Example 7

### Preparation of Desthiobiotin Thiosulfate

This compound was prepared in a similar manner as described in Example 3 by the reaction of 2,3,5,6-tetrafluorophenoxycarbonylmethyl thiosulfate, N,N-diisopropylethylamine salt with the corresponding desthiobiotin amine analog to give an off-white solid. TLC: R_{f}=0.36 (silica gel, chloroform/methanol/acetic acid (75:20:5)). The structure of the resulting desthiobiotin thiosulfate is provided below in Formula (E7).

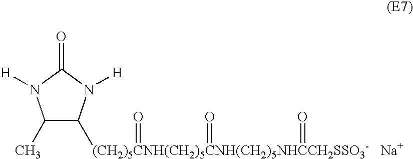

### Example 8

### Preparation of a Bimane Thiosulfate

To a solution of 2,3,5,6-tetrafluorophenoxycarbonylmethyl thiosulfate, N,N-diisopropylethylamine salt was added the corresponding bimane amine analog to give a yellow solid. TLC: R_{f}=0.41 (silica gel, chloroform/methanol/acetic acid (75:20:5)). Absorption maximum: 375 nm in methanol; emission maximum: 458 nm in methanol. The structure of the resulting bimane thiosulfate is provided below in Formula (E8).

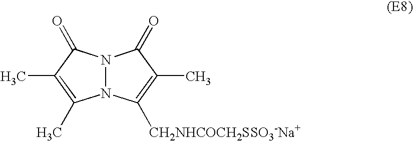

### Example 9

### Preparation of a Fluorescein Thiosulfate

To a solution of sodium thiosulfate pentahydrate (53 mg, 0.21 mmol) in 1 mL of water was added a solution of 5-iodoacetamidofluorescein (100 mg, 0.19 mmol) in 500 μL of ethanol and the mixture was stirred at room temperature for 1 hour. The whole reaction mixture was subjected to LH-20 column (Sephadex) eluting with water to give an orange red solid (60 mg). TLC: R_{f}=0.56 (5% H₂O in CH₃CN). Absorption maximum: 492 nm in pH 9 phosphate buffer; emission maximum: 515 nm in pH 9 phosphate buffer. The structure of the resulting fluorescein thiosulfate is provided below in Formula (E9).

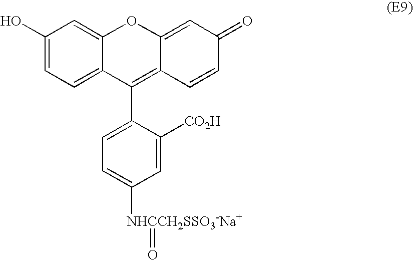

### Example 10

### Preparation of an Alexa Fluor Thiosulfate

2,3,5,6-tetrafluorophenoxycarbonylmethyl thiosulfate, N,N-diisopropylethylamine salt (32 mg, 0.068 mmol) was added to a solution of Alexa Fluor 488 cadaverine, trifluoroacetic acid salt (50 mg, 0.068 mmol; commercially available from Molecular Probes, Inc.; Eugene, Oreg.) and N,N-diisopropylethylamine (12 uL, 0.068 mmol) in 2 mL of water. After stirring for 2 hours at room temperature, the reaction mixture was purified by LH-20 Sephadex column chromatography, with elution with water. The product was converted to its sodium salt by treatment with Dowex 50 WX (sodium form) to give 39 mg of an orange-red solid. TLC: R_{f}=0.25 (silica gel, 20% H₂O in CH₃CN). Absorption maximum: 493 nm in pH 7 phosphate buffer; emission maximum: 518 nm in pH 7 phosphate buffer. The structure of the resulting Alexa Fluor thiosulfate is provided below in Formula (E10).

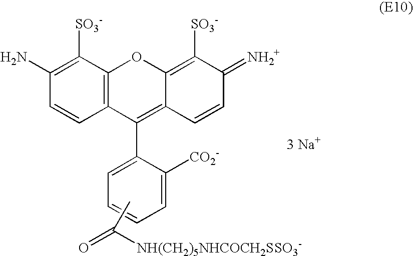

### Example 11

### Reaction of a Boradiazaindacene Thiosulfate with a Thiol

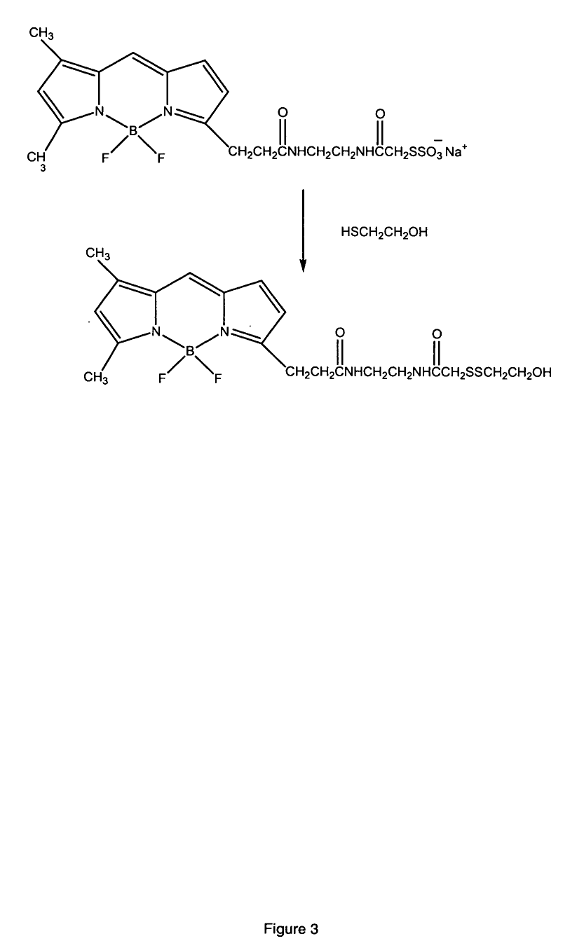

To a solution of boradiazaindacene thiosulfate of example 3 (20 mg, 39 mmol) and 5 mg of sodium acetate in 3 mL of methanol was added 3 μL (42 mmol) of 2-mercaptoethanol and the mixture was stirred at room temperature for 1 hour. The reaction mixture was concentrated in vacuo and the resulting crude product was purified by column chromatography on silica gel, eluting with 10% methanol to give the disulfide as an orange solid. TLC: R_{f}=0.45 (silica gel, 10% methanol in chloroform), MS: m/e 484.39. The reaction is shown in FIG. 3.

### Example 12

### Reaction of a Boradiazaindacene Thiosulfate with a Protein and Demonstration of Reversibility

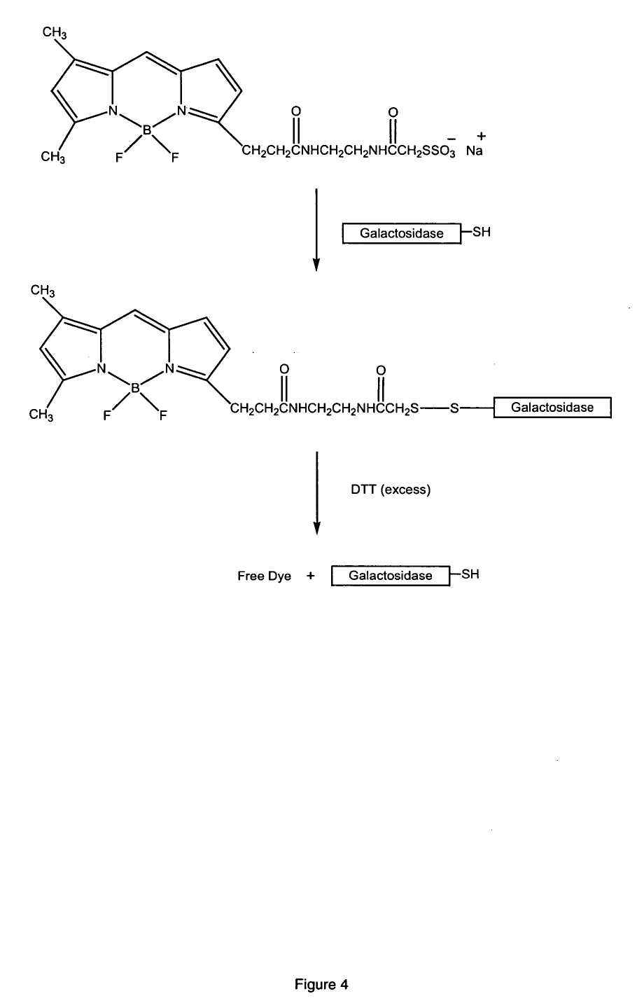

The boradiazaindacene thiosulfate of Example 3 was dissolved at a concentration of 1 mg/mL in water and added to 3 mg of the protein dissolved in a concentration of 10 mg/10 mL in 0.1 M phosphate, 0.1 NaCl, pH 7.5. Compound boradiazaindacene thiosulfate was added at a molar ratio of 30. The reaction was continued for 1 hour at room temperature under argon. The dye-protein conjugate was separated from the unreacted reagent by gel filtration on a CELLUFINE GH-25 column (CELLUFINE is a registered trademark of Chisso Corp.; Osaka, Japan) equilibrated in phosphate buffer solution. The degree of substitution was determined to be 6.2 by measuring the absorbance of the conjugate at 508 nm, using an extinction coefficient of 68,000 cm⁻¹ M⁻¹ for the dye. To demonstrate the reversibility of the product, the dye-protein conjugate was treated with excess dithiothreitol (DTT) and the resulting protein was purified by gel filtration column. The protein obtained showed displacement of dye with a degree of substitution of about 0.20. The reactions are shown in FIG. 4.

All of the compositions and/or methods and/or processes and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and/or processes and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

1. A thiosulfate compound having the formula
R—S—SO₃⁻ or R—S—SO₃H
wherein R is a reporter group, a carrier molecule, or a solid support.

2. The compound of claim 1, wherein the thiosulfate reacts with a thiol group to form a disulfide bond.

3. The compound of claim 1, wherein the reporter group comprises a fluorophore, a phosphorophore, a binding pair member, a polypeptide, a nucleic acid, an enzyme substrate, or a radioisotope tag.

4. The compound of claim 1, wherein the reporter group comprises a coumarin, a xanthene, a cyanine, a pyrene, a borapolyazaindacene, an oxazine, or a bimane.

5. The compound of claim 1, wherein the reporter group is a fluorescent protein.

6. The compound of claim 1, wherein the reporter group is a phycobiliprotein.

7. The compound of claim 1, wherein the reporter group is a cyanine-labeled phycobiliprotein or a xanthene-labeled phycobiliprotein.

8. The compound of claim 1, wherein the reporter group is a peroxidase, a phosphatase, a glycosidase, or a luciferase.

9. The compound of claim 1, wherein the carrier molecule comprises a polypeptide, a protein, a polysaccharide, a carbohydrate, a nucleic acid, a hapten, a psoralen, a drug, a toxin, a hormone, a lipid, a synthetic polymer, a polymeric microparticle, a biological cell, or a virus.

10. The compound of claim 1, wherein the solid support comprises polystyrene, polyacrylic acid, glass, polyol, agarose, cellulose, or dextran.

11. The compound of claim 1, wherein the solid support is a magnetic solid support, a biochip solid support, an organic based bead solid support, or a gel solid support.

12. A thiosulfate compound having the formula:
R-L-S—SO₃⁻ or R-L-S—SO₃H
wherein:
R is a reporter group, a carrier molecule, or a solid support; and
L is a linker.

13. The compound of claim 12, wherein the thiosulfate reacts with a thiol group to form a disulfide bond.

14. The compound of claim 12, wherein L is a substituted alkylene group, an unsubstituted alkylene group, a substituted heteroalkylene group, an unsubstituted heteroalkylene group, a substituted cycloalkylene group, an unsubstituted cycloalkylene group, a substituted heterocycloalkylene group, an unsubstituted heterocycloalkylene group, a substituted arylene group, an unsubstituted arylene group, a substituted heteroarylene group, or an unsubstituted heteroarylene group.

15. The compound of claim 12, wherein L is a substituted (C₁-C₁₅) alkylene group, an unsubstituted (C₁-C₁₅) alkylene group, a substituted 2-10 member heteroalkylene group, or an unsubstituted 2-10-member heteroalkylene group.

16. The compound of claim 12, wherein the reporter group comprises a fluorophore, a phosphorophore, a binding pair member, a polypeptide, a nucleic acid, an enzyme substrate, or a radioisotope tag.

17. The compound of claim 12, wherein the reporter group comprises a coumarin, a xanthene, a cyanine, a pyrene, a borapolyazaindacene, an oxazine, or a bimane.

18. The compound of claim 12, wherein the reporter group is a fluorescent protein.

19. The compound of claim 12, wherein the reporter group is a phycobiliprotein.

20. The compound of claim 12, wherein the reporter group is a cyanine-labeled phycobiliprotein or a xanthene-labeled phycobiliprotein.

21. The compound of claim 12, wherein the reporter group is a peroxidase, a phosphatase, a glycosidase, or a luciferase.

22. The compound of claim 12, wherein the carrier molecule comprises a polypeptide, a protein, a polysaccharide, a carbohydrate, a nucleic acid, a hapten, a psoralen, a drug, a toxin, a hormone, a lipid, a synthetic polymer, a polymeric microparticle, a biological cell, or a virus.

23. The compound of claim 12, wherein the solid support comprises polystyrene, polyacrylic acid, glass, polyol, agarose, cellulose, or dextran.

24. The compound of claim 12, wherein the solid support is a magnetic solid support, a biochip solid support, an organic based bead solid support, or a gel solid support.

25. A thiosulfate crosslinking reagent having the formula:
X-L-S—SO₃⁻ or X-L-S—SO₃H
wherein X is a reactive group; and L is a linker.

26. The crosslinking reagent of claim 25, wherein the thiosulfate reacts with a thiol group to form a disulfide bond.

27. The crosslinking reagent of claim 25, wherein the reactive group is an electrophile, a nucleophile, or a photoactivatable group.

28. The crosslinking reagent of claim 25, wherein the reactive group reacts with an amine, a thiol, or an alcohol.

29. The crosslinking reagent of claim 25, wherein the reactive group reacts with an amine or an alcohol.

30. The crosslinking reagent of claim 25, wherein the reactive group is an activated ester of a carboxylic acid, an acyl azide, an acyl nitrile, an aldehyde, an alkyl halide, an anhydride, an aniline, an aryl halide, an azide, an aziridine, a boronate, a carboxylic acid, a diazoalkane, a halotriazine, a hydrazide, an imido ester, an isocyanate, an isothiocyanate, a phosphoramidite, a reactive platinum complex, or a sulfonyl halide.

31. The crosslinking reagent of claim 25, wherein L is a substituted alkylene group, an unsubstituted alkylene group, a substituted heteroalkylene group, an unsubstituted heteroalkylene group, a substituted cycloalkylene group, an unsubstituted cycloalkylene group, a substituted heterocycloalkylene group, an unsubstituted heterocycloalkylene group, a substituted arylene group, an unsubstituted arylene group, a substituted heteroarylene group, or an unsubstituted heteroarylene group.

32. The crosslinking reagent of claim 25, wherein L is a substituted (C₁-C₁₅) alkylene group, an unsubstituted (C₁-C₁₅) alkylene group, a substituted 2-10 member heteroalkylene group, or an unsubstituted 2-10-member heteroalkylene group.

33. A method for preparing a labeled target analyte, the method comprising:
a) contacting a target analyte comprising a thiol moiety with a compound comprising a reporter group covalently bonded to a thiosulfate moiety; and
b) allowing the thiosulfate moiety to react with the thiol moiety to form a disulfide bond, thereby forming the labeled target analyte.

34. The method of claim 33, wherein the target analyte comprises a polypeptide, a protein, a polysaccharide, a nucleic acid, a carbohydrate, a hapten, a psoralen, a drug, a toxin, a hormone, a lipid, a lipid assembly, a synthetic polymer, a polymeric microparticle, a biological cell, or a virus.

35. The method of claim 33, further comprising, after the allowing step, detecting the presence of the labeled target analyte.

36. The method of claim 33, further comprising, after step b), regenerating the thiol moiety with a reducing agent.

37. The method of claim 33, further comprising, after step b), regenerating the thiol moiety with a thiol compound or a phosphine compound.

38. A method of immobilizing a target analyte, wherein the target analyte comprises a thiol moiety, the method comprising:
a) providing a sample comprising the target analyte;
b) contacting the sample and a solid support covalently bonded to a thiosulfate moiety; and
c) allowing the thiosulfate moiety to react with the thiol moiety of the target analyte to form a disulfide bond, thereby forming the immobilized target analyte.

39. The method of claim 38, further comprising, after the allowing step, separating the sample and the solid support.

40. The method of claim 38, further comprising, after the allowing step, detecting the immobilized target analyte.

41. The method of claim 38, further comprising, after the allowing step, releasing the immobilized target analyte from the solid support using a reducing agent.

42. The method of claim 38, further comprising, after the allowing step, releasing the immobilized target analyte from the solid support using a thiol compound or a phosphine compound.

43. The method of claim 38, wherein the target analyte comprises a polypeptide, a protein, a polysaccharide, a nucleic acid, a carbohydrate, a hapten, a psoralen, a drug, a toxin, a hormone, a lipid, a lipid assembly, a synthetic polymer, a polymeric microparticle, a biological cell, or a virus.

44. A method of immobilizing a target analyte, wherein the target analyte comprises a thiosulfate moiety, the method comprising:
a) providing a sample comprising the target analyte;
b) combining the sample with a solid support comprising a thiol moiety; and
c) allowing the thiol moiety to react with the thiosulfate moiety of the target analyte to form a disulfide bond, thereby forming an immobilized target analyte.

45. The method of claim 44, further comprising, after the allowing step, separating the sample and the solid support.

46. The method of claim 44, further comprising, after the allowing step, detecting the immobilized target analyte.

47. The method of claim 44, further comprising, after the allowing step, releasing the immobilized target analyte from the solid support using a reducing agent.

48. The method of claim 44, further comprising, after the allowing step, releasing the immobilized target analyte from the solid support using a thiol compound or a phosphine compound.

49. The method of claim 44, wherein the target analyte comprises a polypeptide, a protein, a polysaccharide, a nucleic acid, a carbohydrate, a hapten, a psoralen, a drug, a toxin, a hormone, a lipid, a lipid assembly, a synthetic polymer, a polymeric microparticle, a biological cell, or a virus.

50. A kit for use in labeling a target analyte comprising a thiol moiety, the kit comprising a first container comprising a compound comprising a reporter group covalently bonded to a thiosulfate moiety.

51. The kit of claim 50, further comprising a second container containing a reducing agent.

52. The kit of claim 50, further comprising a second container containing a thiol compound or a phosphine compound.

53. The kit of claim 50, further comprising instructions for labeling the target analyte with the compound.

54. A kit for use in immobilizing a target analyte comprising a thiol moiety, the kit comprising a solid support covalently bonded to a thiosulfate moiety.

55. The kit of claim 42, further comprising instructions for immobilizing the target analyte on the solid support.

56. The kit of claim 42, further comprising a container containing a reducing agent.

57. The kit of claim 42, further comprising a container containing a thiol compound or a phosphine compound.

58. A kit for use in immobilizing a target analyte, the kit comprising a crosslinking reagent having the formula:
X-L-S—SO₃⁻ or X-L-S—SO₃H
wherein X is a reactive group; and L is a linker.

59. The kit of claim 58, further comprising a solid support comprising a thiol moiety.

60. The kit of claim 58, further comprising instructions for immobilizing the target analyte to a solid support comprising a thiol moiety.

61. The kit of claim 58, further comprising a container containing a reducing agent.

62. The kit of claim 58, further comprising a container containing a thiol compound or a phosphine compound.
